(19) United States
(12) Patent Application Publication
(10) Pub. No.: US 2015/0150645 A1
(43) Pub. Date: Jun. 4, 2015

(54) OPTICAL SAMPLING OF SURFACE POINTS FOR MEDICAL NAVIGATION

(71) Applicants: Swen Woerlein, Munich (DE); Fritz Vollmer, Munich (DE); Alexander Urban, Forstinning (DE); Martin Mathis, Aying (DE)

(72) Inventors: Swen Woerlein, Munich (DE); Fritz Vollmer, Munich (DE); Alexander Urban, Forstinning (DE); Martin Mathis, Aying (DE)

(21) Appl. No.: 14/391,114

(22) PCT Filed: Apr. 12, 2012
(86) PCT No.: PCT/EP2012/056627

§ 371 Date: Feb. 18, 2015

(51) Int. Cl. A61B 19/00; A61B 5/00

(52) U.S. Cl. CPC A61B 19/54; A61B 5/0064; A61B 2019/5483; A61B 2019/5437

(57) ABSTRACT

A method for sampling surface points on the surface of an object using a medical navigation system and a light beam source, wherein the light beam source generates a light beam which creates a light spot on the surface, and a camera of the medical navigation system records image data from which the location of the light spot is determined as the location of a sample point, said method comprising the steps of: generating a quality measure for a sample point using a quality measurement device which is at least partially attached to the light beam source and used to determine quality measurement data from which the quality measure is determined; and processing the sample point on the basis of the quality measure.

The present invention relates to a method for sampling surface points on the surface of an object using a medical navigation system and a light beam source and to a medical navigation system for sampling surface points on the surface of an object, such as a patient.

Document EP 1 142 536 B1 discloses registering a patient in a medical navigation system using projected light points. A plurality of light points are sequentially generated on the surface of the patient using a light beamer and the location of the light point is determined from the output image of a camera of the medical navigation system. The present invention is based on this prior art.

Using the present invention, the accuracy of the registration process can be increased by evaluating sample points and processing them on the basis of the evaluation result. In particular, this is achieved by the subject-matter of any of the appended independent claims. Advantages, advantageous features, advantageous embodiments and advantageous aspects of the present invention are disclosed in the following and contained in the subject-matter of the dependent claims. Different advantageous features can be combined in accordance with the invention where technically expedient and feasible. In particular, a feature of one embodiment which has the same or a similar function as another feature of another embodiment can be exchanged. In particular, a feature of one embodiment which adds an additional function to another embodiment can be added to said other embodiment.

The present invention relates to a method for sampling surface points on the surface of an object using a medical navigation system and a light beam source, wherein the light beam source generates a light beam which creates a light spot on the surface of the object, and a camera of the medical navigation system records image data from which the location of the light spot is determined as the location of the sample point. The object can be registered in the medical navigation system on the basis of a plurality of sample points, in particular by matching an object dataset which represents the object, in particular as a two-dimensional or three-dimensional image dataset, to the sample points.

In accordance with the method, a measure of the quality of a sample point or “quality measure” for said sample point is generated using a quality measurement device which is at least partially attached to the light beam source and used to determine quality measurement data from which the quality measure is determined. The method also comprises the step of processing the sample point on the basis of the quality measure. In this document, the term “processing” may mean that a sample point is completely ignored, weighted (in particular if object data are matched to the sample points) or amended.

One advantage of the present invention is that at least a part of the quality measurement device is attached to the light beam source, which means that the handling or the status of the light beam source can be used in determining the quality measure and improving the registration result, in particular by excluding or weighting low-quality sample points.

In this document, the term “position” means the (spatial) location in up to three translational dimensions and/or the (rotational) alignment (or angle) in up to three rotational dimensions.

In this document, the terms “light spot” and “sample point” are used, wherein a light spot is an area, in particular an area of the surface of the object, which is illuminated by the light beam generated by the light beam source, and a sample point represents a location of the light spot, preferably in up to three translational/spatial dimensions. Unless indicated otherwise and/or necessarily not the case, the terms “light spot” and “sample point” are used synonymously.

It has been found that a number of factors may impair the quality of a sample point, such that a quality measure accordingly has to be determined. Depending on the factor or factors to be considered, one or more of the embodiments described below may be suitable.

Such factors include the speed (or velocity) of the light beam source and therefore of the light spot on the surface of the object, the distance between the object and the light beam source, the shape of the light spot and the material which reflects the light beam. In the latter case, the light beam may hit an obstacle rather than the object to be registered. It is thus advantageous if the quality measure reflects the fact that the light beam has hit an obstacle rather than the surface of the object. In this document, the term “foreign material” is used to describe any material which does not form the surface of the object to be registered. If, for example, the object to be registered is a patient's head, then the surface of the object is typically the skin. However, items such as intubation tubes, headrests, hair, plasters or the like do not belong to the object and are therefore regarded as obstacles comprising foreign material.

In one embodiment, the quality measurement device comprises a distance measurement device attached to the light beam source, and the distance between the light beam source and the light spot on the object is determined using the distance measurement device. The measured distance can then be used in several ways.

In one implementation, the quality measure is based on a comparison of the measured distance with at least one of a minimum threshold distance, a maximum threshold distance and the measured distance of the temporally preceding sample point. A minimum threshold distance represents a minimum distance which the light beam source should exhibit from the surface of the object. If the measured distance is below the minimum threshold distance, then the quality of the sample point is regarded as being correspondingly lower, thus resulting in a reduced quality measure. If the measured distance is smaller than the minimum threshold distance, the light spot may for example be too small to be detected. The maximum threshold distance represents a distance between the light beam source and the light spot on the surface which should not be exceeded. If the measured distance is larger than the maximum threshold distance, the quality of the sample point is again regarded as being correspondingly lower, thus resulting in a reduced quality measure. If the measured distance exceeds the maximum threshold distance, the light spot on the surface may be too large to be detected to a desired level of accuracy.

The last aspect including the distance of the temporally preceding sample point relates to the measured distance for two temporally consecutive sample points. If there is a sudden change in the measured distances, such that the difference between the measured distances of two consecutive sample points is above a predetermined threshold, then it is assumed that the light beam has hit a foreign material. If the measured distance of the former sample point is larger than the measured distance of the latter sample point, then it is assumed that the light spot corresponding to the latter sample point lies on the foreign material. If the measured distance of the former light spot is smaller than the measured distance of the latter light spot, then it is assumed that the former light spot lies on the foreign material.

The distance measurement device is optionally configured to detect modulated light reflected back from the surface of the object (or an obstacle) in order to calculate the distance. The reflected light can be analysed for brightness deviations. By assessing the brightness or brightness ratio, it is possible to distinguish between the surface of the object and an obstacle. It is possible to calibrate the distance measurement device to the reflectivity of the surface of the object.

In another implementation, the quality measurement device comprises a marker device attached to the light beam source, wherein an additional sample point is determined from the position of the marker device, the direction of the light beam relative to the marker device and the distance of the light beam source from the light spot on the object. In other words, the sample point is determined by detecting the light spot, while the additional sample point is determined—at the same time as the sample point and/or at the same position of the light beam source relative to the object—by tracking the light beam source itself. The direction of the light beam relative to the marker device attached to a light beam source is known. If the position of the marker device at the distance between the light spot and the light beam source is known, then the location of the additional sample point can be determined.

The quality measure of the sample point is then determined from the distance between the location of the sample point and the location of the additional sample point. If, for example, the distance is below a predetermined threshold value, then the quality measure is set accordingly.

The term “processing” preferably means amending the location of the sample point on the basis of the location of the additional sample point. The amended sample point is for example the (weighted) average of the sample point and the additional sample point. It should be noted that the step of determining the quality measure can be omitted, such that the location of the sample point is amended directly, without the intermediate step of determining the quality measure. Directly amending the location of the sample point is equivalent to determining and using the quality measure.

It is the function of a marker to be detected by a marker detection device (for example, a camera or an ultrasound receiver), such that its spatial position (i.e. its spatial location and/or alignment) can be ascertained. The detection device is in particular part of a navigation system. The markers can be active markers. An active marker can for example emit electromagnetic radiation and/or waves, wherein said radiation can be in the infrared, visible and/or ultraviolet spectral range. The marker can also however be passive, i.e. can for example reflect electromagnetic radiation in the infrared, visible and/or ultraviolet spectral range. To this end, the marker can be provided with a surface which has corresponding reflective properties. It is also possible for a marker to reflect and/or emit electromagnetic radiation and/or waves in the radio frequency range or at ultrasound wavelengths. A marker preferably has a spherical and/or spheroid shape and can therefore be referred to as a marker sphere; markers can also, however, exhibit a cornered—for example, cubic—shape.

Preferably, markers attached to the light beam source are passive markers and the camera for detecting the markers is the same camera as the camera for detecting the light spot on the surface of the object.

A marker device can for example be a reference star or a pointer or one or more (individual) markers in a predetermined spatial relationship. A marker device comprises one, two, three or more markers in a predetermined spatial relationship. This predetermined spatial relationship is in particular known to a navigation system and for example stored in a computer of the navigation system.

In accordance with another embodiment of the present invention, the quality measurement device comprises a marker device attached to the light beam source, and the method comprises the additional step of pre-registering the object, wherein the quality measure is based on the angle between the normal vector on the surface of the object at the location of the light spot and the direction of the light beam. During pre-registering, the object is approximately registered. Pre-registering can be performed by determining a (reduced) set of sample points which represent landmarks of the object. Based on the sample points corresponding to the landmarks, object data representing the object, such as image data as explained above, can be matched to the sample points. After pre-registering, an approximate position of the object is known to the medical navigation system. This means that the normal vector on the surface of the object at the location of the light spot is also (approximately) known to the medical navigation system. In an ideal configuration, the normal vector and the direction of the light beam coincide. If this is not the case, the shape of the light spot is distorted, such that the shape of the light spot differs from the cross-section of the light beam. A large distortion, in which the angle between the normal vector and the direction of the light beam exceeds a predetermined threshold, reduces the quality of the sample point, such that the quality measure of the sample point has to be reduced.

A landmark is a defined element of an anatomical body part which is always identical or recurs with a high degree of similarity in the same anatomical body part of multiple patients. Typical landmarks are for example the epicondyles of a femoral bone or the tips of the transverse processes and/or dorsal process of a vertebra. The points (main points or auxiliary points) can represent such landmarks. A landmark which lies on (in particular, on the surface of) a characteristic anatomical structure of the body part can also represent said structure. The landmark can represent the anatomical structure as a whole or only a point or part of it. A landmark can also for example lie on the anatomical structure, which is in particular a prominent structure. An example of such an anatomical structure is the posterior aspect of the iliac crest. Other landmarks include a landmark defined by the rim of the acetabulum, for instance by the centre of the rim. In another example, a landmark represents the bottom or deepest point of an acetabulum, which is derived from a multitude of detection points. Thus, one landmark can in particular represent a multitude of detection points. As mentioned above, a landmark can represent an anatomical characteristic which is defined on the basis of a characteristic structure of the body part. Additionally, a landmark can also represent an anatomical characteristic defined by a relative movement of two body parts, such as the rotational centre of the femur when moved relative to the acetabulum.

In another embodiment, the quality measurement device comprises a marker device attached to the light beam source, wherein the speed of the light beam source is determined by tracking the marker device and the quality measure is determined on the basis of the determined speed. This utilises the fact that the camera of the medical navigation system outputs a sequence of images at a certain frame rate and a certain exposure time for each image or frame. If the light spot moves by more than a predetermined maximum distance on the surface of the object during the exposure time, then the shape of the light spot in the image captured by the camera differs from the cross-section of the light beam by too much, thus impairing the determination of the sample point. It is thus advantageous to set the quality measure as a function of the determined speed of the light beam source and optionally also as a function of the distance between the surface of the object and the light beam source. In this context, the term “speed” may include a translational velocity, in particular perpendicular to the direction of the light beam, and/or an angular velocity, in particular about an axis perpendicular to the direction of the light beam.

In another embodiment, the quality measurement device comprises an acceleration sensor, and the quality measure is determined on the basis of the acceleration data which are outputted by the acceleration sensor and represent the acceleration of the light beam source. The acceleration sensor is configured to determine the acceleration of the light beam source. By taking into account the acceleration, which is preferably represented by acceleration data, the speed of the light beam source can be determined. The advantages of this embodiment are the same as those explained above with reference to the speed determined using a marker device. However, the acceleration itself can be used to determine the quality measure.

In another embodiment, the quality measurement device comprises an optical properties detector for determining the optical properties, such as the brightness, the polarisation, the spectrum and/or the frequency (shift), of the light spot on the surface of the object, and the quality measure is determined on the basis of the optical properties of the light spot. Regarding the brightness, this utilises the fact that the reflectivity of foreign material is different to the reflectivity of the surface of the object. If the brightness changes between two temporally consecutive sample points, or is below a minimum threshold brightness or above a maximum threshold brightness depending on the reflectivity of the surface of the object, then a light spot is regarded as lying on an obstacle consisting of foreign material. The quality measurement device preferably also comprises a distance measurement device, such that the criterion of brightness can be replaced by a criterion of the reflectivity of the surface at the light spot, which means that an absolute property of the surface of the object can be used. Preferably, the brightness detector is calibrated to the object, for example by pointing the light beam at the object such that the light spot is on the surface of the object and detecting the brightness of the light spot as a reference brightness.

Further, different materials may expose different reflection properties regarding the polarisation of the light beam, so if the polarisation of the reflected light beam is analysed, then conclusions regarding the material can be drawn from this analysis. The frequency of the reflected light beam, or the frequency shift (Doppler shift) of the reflected light beam compared to the generated light beam, can also be used for determining the movement speed of the light beam source. Further, the material can be determined from the spectrum (or colour) of the reflected light beam.

In another embodiment, the quality measurement device comprises a camera, wherein the image data provided by the camera are preferably processed by image processing in order to detect foreign material in the output image, and the quality measure is determined on the basis of whether the light spot lies on foreign material or not. Preferably, the centre of the field of view of the camera is directed in the same direction as the light beam. Using image processing, the image provided by the camera can be segmented, at least into the object to be registered and an obstacle comprising foreign material. If the light spot is found to be lying on foreign material, such that the light beam is reflected by an obstacle, then the quality measure of the sample point is set accordingly. The camera can be used to detect the optical properties of the light spot.

It should be noted that the camera is not necessarily attached to the light beam source. The camera can be provided independently, for example as a part of the camera of the medical navigation system. A camera which is not attached to the light beam source is also referred to as an external camera. Such an external camera can be combined with any one or more of the quality measurement devices attached to the light beam source as explained above. However, an external camera alone can also be used as the quality measurement device.

In yet another embodiment of the present invention, the method comprises the additional step of providing feedback information based on the quality measure of the sample point. The feedback information can be acoustic, visual or tactile in nature. The feedback information can for example be provided by modulating the light beam of the light beam source, in particular by switching the light beam on and off and/or by changing the colour of the light beam. Using this feedback information, it is possible to indicate to the operator of the light beam source that the quality measure of the currently sampled sample point is below a predetermined threshold value.

It should be noted that any data are preferably represented by a corresponding dataset, i.e. image data are for example represented by an image dataset, quality measurement data by a quality measurement dataset, and so on.

The present invention also relates to a medical navigation system for sampling surface points on the surface of an object, said system comprising a camera, a light beam source and a control unit. The light beam source is configured to generate a light beam which generates a light spot on the surface of the object, and the camera is configured to record image data which represent the light spot. The control unit is configured to determine the location of the light spot as the location of a sample point from the image data captured by the camera. The medical navigation system also comprises a quality measurement device which is at least partially attached to the light beam source and is used to determine quality measurement data. The control unit is configured to generate a quality measure for a sample point on the basis of the quality measurement data and to process the sample point on the basis of the quality measure. The advantages of this configuration are the same as those described above for the method according to the present invention.

The quality measurement device preferably comprises at least one of a distance measurement device, a marker device, an acceleration sensor and a camera. The control unit is also preferably adapted to carry out the method steps explained above.

The method in accordance with the invention is in particular a data processing method. The data processing method is preferably performed using technical means, in particular a computer such as a medical navigation system or a part of a medical navigation system. In particular, the data processing method is executed by or on the computer. The computer in particular comprises a processor and a memory in order to process the data, in particular electronically and/or optically. The calculating steps described are in particular performed by a computer. Determining steps or calculating steps are in particular steps of determining data within the framework of the technical data processing method, in particular within the framework of a program. A computer is in particular any kind of data processing device, in particular electronic data processing device. A computer can be a device which is generally thought of as such, for example desktop PCs, notebooks, netbooks, etc., but can also be any programmable apparatus, such as for example a mobile phone or an embedded processor. A computer can in particular comprise a system (network) of “sub-computers”, wherein each sub-computer represents a computer in its own right. The term “computer” also encompasses a cloud computer, in particular a cloud server. The term “cloud computer” also encompasses a cloud computer system which in particular comprises a system of at least one cloud computer, in particular a plurality of operatively interconnected cloud computers such as a server farm. The cloud computer is preferably connected to a wide area network such as the world wide web (WWW). Such a cloud computer is located in a so-called cloud of computers which are all connected to the world wide web. Such an infrastructure is used for “cloud computing” which describes computation, software, data access and storage services which do not require the end user to know the physical location and configuration of the computer which delivers a specific service. In particular, the term “cloud” is used as a metaphor for the internet (world wide web). The cloud in particular provides computing infrastructure as a service (IaaS). The cloud computer may function as a virtual host for an operating system and/or data processing application which is used for executing the method of the invention. The cloud computer is preferably an Elastic Compute Cloud (EC2) as provided by Amazon Web Services™. A computer in particular comprises interfaces in order to receive or output data and/or perform an analogue-to-digital conversion. The data are in particular data which represent physical properties and/or are generated from technical signals. The technical signals are in particular generated by means of (technical) detection devices (such as for example devices for detecting marker devices) and/or (technical) analytical devices (such as for example devices for performing imaging methods), wherein the technical signals are in particular electrical or optical signals. The technical signals represent in particular the data received or outputted by the computer.

The invention also relates to a program which, when running on a computer or when loaded onto a computer, causes the computer to perform one or more or all of the method steps described herein and/or to a program storage medium on which the program is stored (in particular in a non-transitory form) and/or to a computer on which the program is running or into the memory of which the program is loaded and/or to a signal wave, in particular a digital signal wave, carrying information which represents the program, in particular the aforementioned program, which in particular comprises code means which are adapted to perform any or all of the method steps described herein.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, in particular computer-readable data storage medium comprising computer-usable, in particular computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, in particular a data processing device comprising a digital processor (central processing unit (CPU)) which executes the computer program elements and optionally a volatile memory (in particular, a random access memory (RAM)) for storing data used for and/or produced by executing the computer program elements. Within the framework of the present invention, a computer-usable, in particular computer-readable data storage medium can be any data storage medium which can include, store, communicate, propagate or transport the program for use on or in connection with the instruction-executing system, apparatus or device. The computer-usable, in particular computer-readable data storage medium can for example be, but is not limited to, an electronic, magnetic, optical, electromagnetic, infrared or semiconductor system, apparatus or device or a medium of propagation such as for example the internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can in particular include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or vibration element incorporated into an instrument).

As an alternative, the quality measurement device is completely separated from the light beam source. An example of such a quality measurement device is a camera, such as a tracking camera of the medical navigation system or any other camera, above referred to as an external camera. The camera captures an image which contains the light spot as the quality measurement data and determines the quality measure by analyzing the quality measurement data. The determination can for example be based on one or more of the speed, the size, the shape, the intensity (brightness), the frequency or the polarization of the light spot as explained above. Further, the camera image can be analyzed in order to determine whether or not the light spot is located on the surface of the object or on foreign material as described above. Preferably, the quality measure is used for a gating process which interrupts the sampling of surface points if the quality measure is below a predetermined threshold.

It should be noted that the present invention does not relate to or comprise any surgical or therapeutic step. Instead, the present invention only relates to analysing light spots on an object and determining quality measures of sample points determined from the light spot.

The present invention shall now be explained in more detail with reference to the accompanying drawings. The figures show:

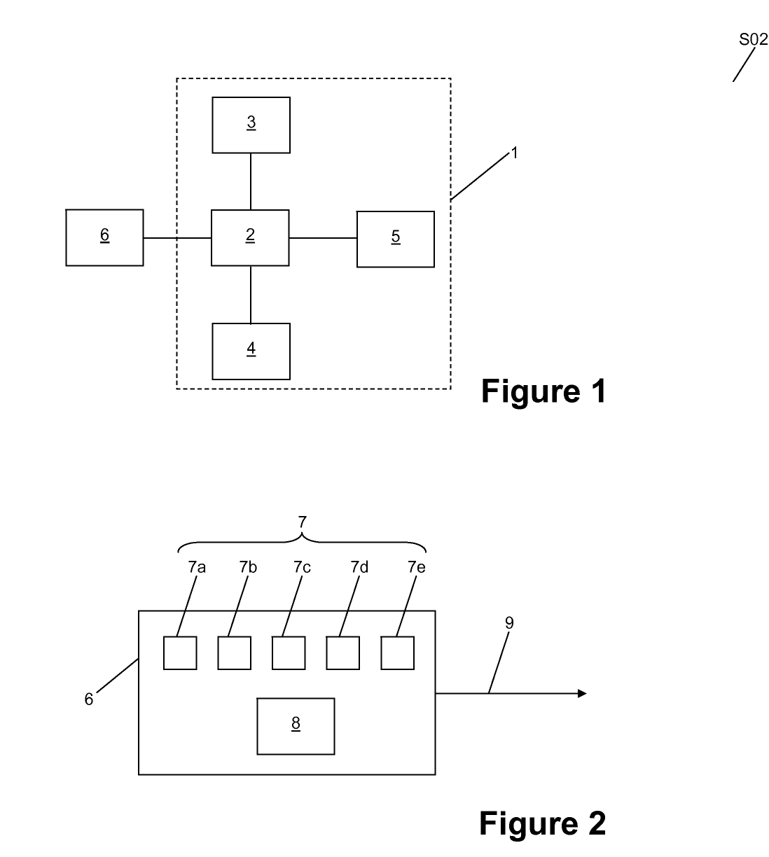

FIG. 1 a schematic representation of a medical navigation system and a light beam source;

FIG. 2 a schematic representation of a light beam source;

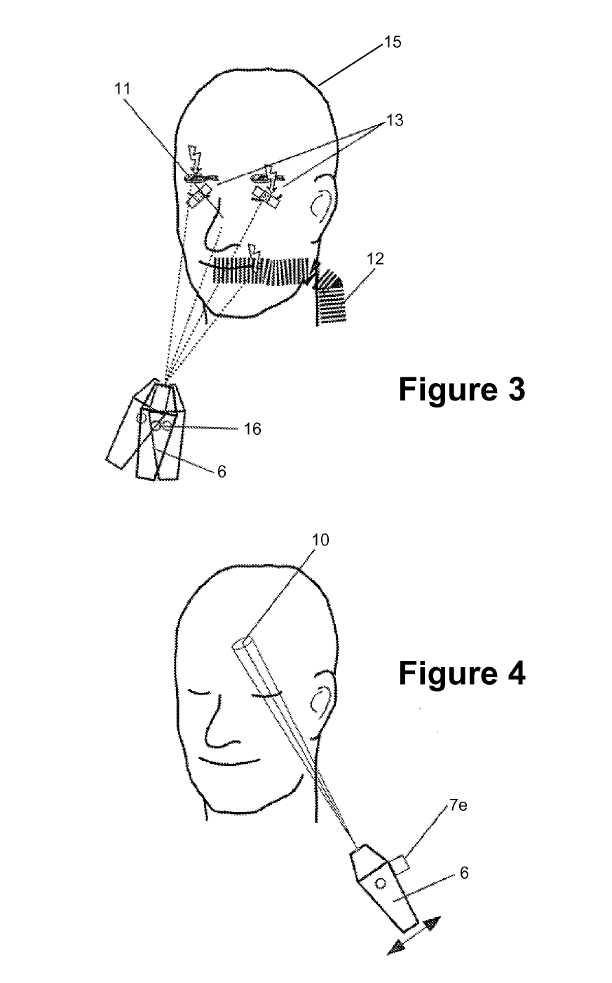

FIG. 3 a configuration comprising a laser beam source which includes a distance sensor and a brightness sensor;

FIG. 4 a configuration comprising a light beam source which includes an acceleration sensor;

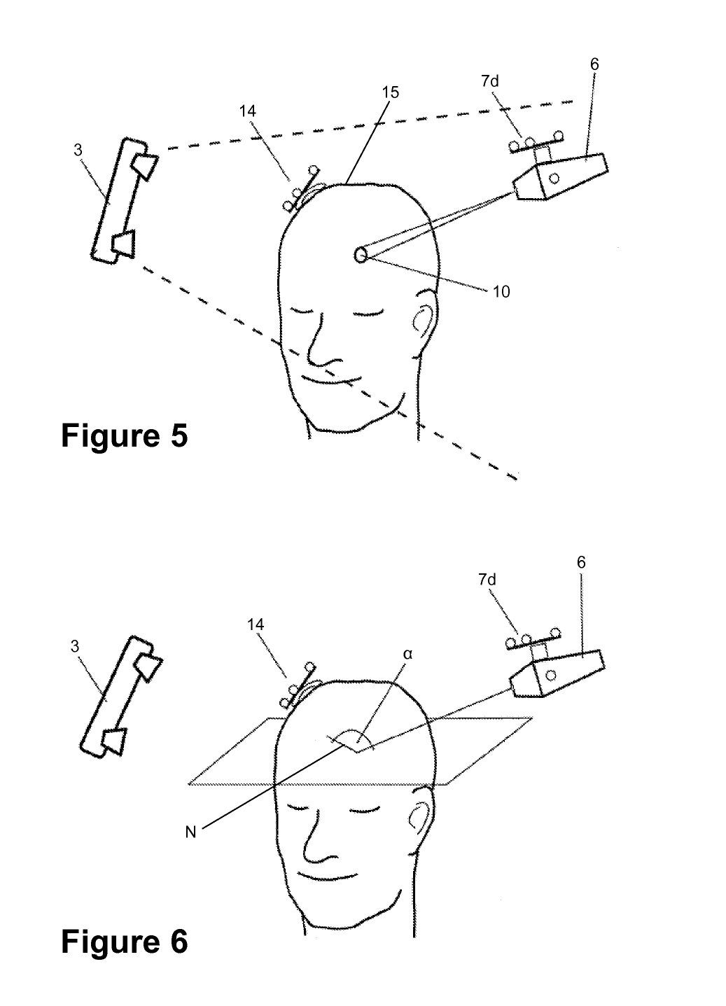

FIG. 5 a configuration comprising a light beam source which includes a marker device; and

FIG. 6 another configuration comprising a light beam source which includes a marker device.

FIG. 1 schematically shows the structure of a medical navigation system 1 and a light beam source 6. The medical navigation system 1 comprises a central processing unit 2 connected to a stereoscopic camera 3, a display device 4 and an input device 5. The stereoscopic camera 3 is arranged in a fixed position. The input device 5, which may for example be a keyboard, a trackball, a mouse or a touch screen, is used to input data into the medical navigation system 1. The display device 4, which may for example be a monitor, is used to display information of the medical navigation system 1. The light beam source 6 may also be regarded as part of the medical navigation system 1.

A data connection is optionally provided between the light beam source 6 and the medical navigation system 1, depending on whether or not data are to be sent from the light beam source 6 to the medical navigation system 1 and/or vice versa. In particular, the data connection is required if an active device, i.e. a device which generates data, is attached to the light beam source 6 and has to send the acquired data to a medical navigation system 1.

FIG. 2 schematically shows the structure of the light beam source 6. In the present example, the light beam source 6 is a laser beam source. The light beam source 6 comprises a laser diode 8 which generates a laser beam 9, wherein the direction of the laser beam 9 relative to the light beam source 6 is known. In this embodiment, the laser diode 8 generates a laser beam of two different wavelengths. One wavelength lies within the infrared spectrum and creates a light spot on the surface of an object, wherein the light spot is to be detected using the camera 3 of the medical navigation system 1. The other wavelength lies within the visible spectrum, for example corresponding to red light, and is used to create a visible light spot to be aimed at a target, in particular the object, using the light beam source 6. The light spots of the visible and infrared light preferably coincide and the visible and infrared light beams are preferably also congruent. Optionally, two different laser diodes generate the visible and infrared light beams, respectively.

When operated, the light beam source 6 is used to create a light spot 10 on the surface of an object 15 (see FIGS. 3 to 6). The stereoscopic camera 3 of the medical navigation system 1 captures a three-dimensional image which represents the light spot 10 and at least a part of the object 15. The image captured by the camera 3 is analysed by the central processing unit 2 in order to locate the light spot 10. Locating the light spot 10 means determining the location (in up to three translational dimensions) of the light spot 10, thus resulting in up to three determined co-ordinates of the light spot 10. The location of the light spot 10 is used as the location of the sample point on the surface of the object 15. Once a plurality of sample points have been acquired, the object 15 can be registered by the medical navigation system 1. In one implementation of the registration process, a three-dimensional image dataset of the object 15 is matched to the sample points.

In accordance with the present invention, a sample point is processed on the basis of a quality measure which corresponds to the sample point. The quality measure represents the quality or reliability of a sample point. The quality of a sample point can depend on several factors, such as the distance between the light beam source 6 and the object 15 and/or the relative position between the light beam source 6 and the object 15 or an obstacle in the path of the light beam 9 if the light spot 10 lies on an obstacle and not on the object 15.

The quality measure is determined using a quality measurement device 7 which is at least partly attached to the light beam source 6. In accordance with FIG. 2, at least one of a brightness sensor 7a, a distance sensor 7b, a camera 7c, a marker device 7d and an acceleration sensor 7e is attached to the light beam source 6. The brightness sensor 7a is configured to detect the brightness of the reflected light beam 9. The distance sensor 7b is configured to detect the distance between the light beam source 6 and the object 15. The camera 7c is configured to generate image data, wherein the optical axis of the camera 7c preferably coincides with or is parallel to the direction of the light beam 9. The marker device 7d is a set of markers, preferably comprising at least three markers, in a known spatial relationship. The camera 3 of the navigation system 1 captures an image from which the central processing unit 2 can determine the locations of the markers and, from these locations, the position of the marker device 7d. Since the position of the light beam source 6 relative to the marker device 7d is typically known, the central processing unit 2 can therefore determine the position of the light beam source 6. The acceleration sensor 7e is configured to detect the acceleration of the light beam source 6. By taking into account the acceleration over time, in particular by calculating the integral, it is possible to calculate the speed of the light beam source 6.

If at least one of a brightness sensor 7a, a distance sensor 7b, a camera 7c and an acceleration sensor 7e is attached to the light beam source 6, then the light beam source 6 preferably also comprises a data transmission unit (not shown) for transmitting the data acquired by one or more of these devices to a central processing unit 2. The data connection can be wireless, for example using a known standard such as Bluetooth, or can be realised by a wire connection.

FIG. 3 shows a configuration in which the light beam source 6 comprises a brightness sensor 7a and an optional distance sensor 7b. In this configuration, as in the following configurations, the object 15 to be registered is a patient's head. An intubation tube 12 leads into the patient's mouth and plasters 13 have been used to keep the patient's eyes closed.

When operated, the light beam source 6 is manipulated in such a way that the light spot 10 of the light beam 9 moves over the surface of the object 15. As explained above, the location of the light spot 10 is detected using the camera 3 and the central processing unit 2 of the navigation system 1. The brightness sensor 7a detects the brightness of the light spot 10, i.e. the brightness of the reflected light beam 9, in particular the brightness of a wavelength within the infrared spectrum. The brightness is represented by brightness data and transmitted to a central processing unit 2. On the basis of the transmitted brightness data, the central processing unit 2 determines whether the light spot 10 lies on the skin of the object 15, such as at the position 11, or on foreign material such as the material of the intubation tube 12, the plasters 13 or hair, such as for example the patient's eyebrows. If the light spot 10 lies on the skin, then an appropriate quality measure for the corresponding sample point is set, for example an absolute value such as for example 1. If the light spot 10 lies on an obstacle, then a low quality measure is set, such as for example 0. When registering the object 15, sample points with a quality measure of 0 are preferably disregarded.

If a light beam source 6 also comprises a distance sensor 7b, such as for example a laser range finder, then the brightness detected by the brightness sensor 7a can be scaled to the measured distance. This allows any loss of brightness due to the length of the light beam path from the light beam source 6 to the object 15 and back to be compensated for.

The light beam source 6 also comprises an optional calibration button 16. In order to perform a calibration, the light beam source 6 is positioned such that the light spot 10 lies on the surface of the object 15. The calibration button 16 is then activated, and the central processing unit 2 determines the brightness of the light spot 10 at the time the calibration button 16 was activated. This brightness is used as a reference brightness for determining whether or not the light spot 10 lies on the surface of the object 15. A brightness range either side of the reference brightness is for example defined, wherein the light spot 10 is regarded as lying on the surface of the object 15 if a measured brightness lies within this range and is regarded as lying on an obstacle if the measured brightness lies outside this range.

The measured distance determined by the distance sensor 7b can additionally or alternatively be used to determine quality measurement data. In one example, the quality measure can be set such that it is inversely proportional to the absolute value of the difference between the measured distance and a reference distance, i.e. the quality measure is decreased if the distance between the light beam source 6 and the object 15 is too small or too large. The measured distances for two temporally consecutive sample points can also optionally be compared. If the difference between the two measured distances is above a predetermined threshold, then one of the sample points is regarded as corresponding to the surface of an obstacle. In FIG. 3, obstacles are indicated by lightning bolts.

FIG. 4 schematically shows a configuration in which an acceleration sensor 7e is attached to the light beam source 6. In a typical camera 3, an image is captured over a certain exposure time. If the light spot 10 moves on the surface of the object 15 during the exposure time, the shape of the light spot 10 in the camera image does not equal the cross-section of the light beam 9, but rather has a different shape according to the movement of the light beam source 6. In the configuration of FIG. 4, the acceleration sensor 7e detects the acceleration of the light beam source 6 and transmits the acquired acceleration data to the central processing unit 2. On the basis of the acceleration data, the central processing unit 2 calculates the amount of movement by the light spot 10 on the surface of the object 15 during the exposure time of one image captured by the camera 3. The central processing unit 2 then sets the quality measure of the sample point corresponding to the light spot 10 in accordance with the determined movement. The quality measure is in particular set to 0 if the determined movement exceeds a predetermined threshold and set to 1 if the determined movement does not exceed a predetermined threshold.

Instead of or in addition to a brightness sensor 7a, a camera 7c can be attached to the light beam source 6. The image captured by the camera 7c is transmitted to the central processing unit 2 which analyses the image and detects obstacles. The central processing unit 2 also determines whether the light spot 10 lies on the surface of the object 15 or on an obstacle.

The configurations shown in FIGS. 5 and 6 represent preferred embodiments. In these configurations, a marker device 7d is attached to the light beam source 6.

In one variant of these embodiments, a distance sensor 7b is also attached to the light beam source 6. The sample point is acquired by detecting the light spot 10 on the surface of the object 15 using the camera 3 and the central processing unit 2 of the medical navigation system 1, as explained above. An additional sample point is also acquired by determining the position of the marker device 7d, and therefore the position of the light beam source 6, using the camera 3 and the central processing unit 2 of the medical navigation system 1 and the distance measured using the distance sensor 7b. On the basis of the position of the light beam source 6 and the distance of the light spot 10 from the light beam source 6, the central processing unit 2 calculates the location of the additional sample point. The central processing unit 2 then calculates the quality measure as a function of the distance between the locations of the sample point and the additional sample point. The central processing unit 2 optionally amends the location of the sample point by averaging the locations of the sample point and the additional sample point.

In another variant of the embodiments, as shown with reference to the configuration in FIG. 6, the object 15 is pre-registered to the medical navigation system 1, for example using a marker device 14 attached to the object 15 or by sampling a reduced number of sample points, for example landmarks, of the object 15. Once pre-registration is complete, the position of the object 15 is approximately known to the medical navigation system 1. This means that the orientation of the normal vectors N onto the surface of the object 15 is also approximately known. The central processing unit 2 determines the direction of the light beam 9 from the position of the marker device 7d attached to the light beam source 6 and determines the direction of the normal vector N on the surface of the object 15 at the location of the light spot 10. The central processing unit 2 then compares the direction of the light beam 9 and the direction of the normal vector N, for example by calculating an angle α between the normal vector N and the light beam 9. The central processing unit 2 then sets the quality measure of the sample point corresponding to the light spot 10 in accordance with the result of this comparison. The quality measure is in particular set such that it is inversely proportional to the difference or the angle α. It is thus possible to compensate for the fact that a light beam falling obliquely onto a surface creates a distorted light spot. If, for example, the cross-section of the light beam 9 is circular and the direction of the normal vector N and the direction of the light beam 9 are not identical, then the shape of the light spot 10 might be elliptical, which can impair the accuracy of the sample point.

The medical navigation system can preferably provide a feedback signal to the user of the light beam source 6, depending on the determined quality measure of the sample point. This feedback information can be acoustic, visual or tactile in nature. The medical navigation system 1 can for example output a warning sound if the quality measure of the sample point is below a predetermined threshold. The medical navigation system could also output a warning on the display device 4 or change the light beam, for example by changing the intensity at the visual wavelength (for example, switching the visible wavelength on and off) or change the colour of the visible wavelength. The light beam source 6 could provide feedback tactilely by vibrating.

1. A method for sampling surface points on the surface of an object using a medical navigation system and a light beam source, wherein the light beam source generates a light beam which creates a light spot on the surface, and a camera of the medical navigation system records image data from which the location of the light spot is determined as the location of a sample point, said method comprising the steps of:
generating a quality measure for a sample point using a quality measurement device which is at least partially attached to the light beam source and used to determine quality measurement data from which the quality measure is determined; and
processing the sample point on the basis of the quality measure.

2. The method according to claim 1, wherein the quality measurement device comprises a distance measurement device attached to the light beam source, and the distance between the light beam source and the light spot on the object is determined using the distance measurement device.

3. The method according to claim 2, wherein the quality measure is based on a comparison of the measured distance with at least one of a minimum threshold distance, a maximum threshold distance and the measured distance of the temporally preceding sample point.

4. The method according to claim 2, wherein:
the quality measurement device comprises a marker device attached to the light beam source;
an additional sample point is determined from the position of the marker device, the direction of the light beam relative to the marker device and the distance of the light beam source from the light spot on the object; and
the quality measure of the sample point is determined from the distance between the location of the sample point and the location of the additional sample point.

5. The method according to claim 4, wherein the term “processing” means amending the location of the sample point on the basis of the location of the additional sample point.

6. The method according to claim 1, wherein the quality measurement device comprises a marker device attached to the light beam source, and the method comprises the additional step of pre-registering the object, wherein the quality measure is based on the angle between the normal vector on the surface of the object at the location of the light spot and the direction of the light beam.

7. The method according to claim 6, wherein:
the quality measurement device comprises a marker device attached to the light beam source;
the speed of the light beam source is determined by tracking the marker device; and
the quality measure is determined on the basis of the determined speed.

8. The method according to claim 1, wherein the quality measurement device comprises an acceleration sensor, and the quality measure is determined on the basis of the acceleration data which are outputted by the acceleration sensor and represent the acceleration of the light beam source.

9. The method according to claim 1, wherein the quality measurement device comprises an optical properties detector for determining the optical properties, such as the brightness, of the light spot on the surface of the object, and the quality measure is determined on the basis of the properties of the light spot.

10. The method according to claim 1, wherein:
the quality measurement device comprises a camera, the image data of the camera are processed by image processing in order to detect foreign material in the output image; and
the quality measure is determined on the basis of whether the light spot lies on foreign material or not.

11. The method according to claim 1, comprising the additional step of providing feedback information based on the quality measure of the sample point.

12. A computer program embodied on a non-transitory computer readable medium which, when running on a computer or when loaded onto a computer, causes the computer to sample surface points on the surface of an object using a medical navigation system and a light beam source, wherein the light beam source generates a light beam which creates a light spot on the surface, and a camera of the medical navigation system records image data from which the location of the light spot is determined as the location of a sample point, comprising the steps of:
generating a quality measure for a sample point using a quality measurement device which is at least partially attached to the light beam source and used to determine quality measurement data from which the quality measure is determined; and
processing the sample point on the basis of the quality measure.

13. A medical navigation system for sampling surface points on the surface of an object, said system comprising a camera, a light beam source and a control unit, wherein the light beam source is configured to generate a light beam which creates a light spot on the surface, the camera is configured to record image data and the control unit is configured to determine the location of the light spot, as the location of a sample point, from the image data, the system further comprising a quality measurement device which is at least partially attached to the light beam source and used to determine quality measurement data, wherein the control unit is also configured to determine a quality measure for a sample point on the basis of the quality measurement data and to process the sample point on the basis of the quality measure.

14. The medical navigation system according to claim 13, wherein the quality measurement device comprises at least one of a brightness measurement device, a distance measurement device, a marker device, an acceleration sensor and a camera.

15. The medical navigation system according to claim 13, wherein the control unit is adapted to sample surface points on the surface of an object using the medical navigation system and a light beam source, wherein the light beam source generates a light beam which creates a light spot on the surface, and a camera of the medical navigation system records image data from which the location of the light spot is determined as the location of a sample point, comprising the steps of:
generating a quality measure for a sample point using a quality measurement device which is at least partially attached to the light beam source and used to determine quality measurement data from which the quality measure is determined; and
processing the sample point on the basis of the quality measure.
